(19) United States
(12) Patent Application Publication
(10) Pub. No.: US 2016/0089064 A1
(43) Pub. Date: Mar. 31, 2016

(54) LIVING BODY MEASURING LIGHT SOURCE SYSTEM AND LIVING BODY MEASUREMENT APPARATUS

(71) Applicant: Seiko Epson Corporation, Tokyo (JP)

(72) Inventor: Kazuhiro NISHIDA, Matsumoto (JP)

(21) Appl. No.: 14/859,724

(22) Filed: Sep. 21, 2015

(30) Foreign Application Priority Data
Sep. 26, 2014 (JP) 2014-197033

(51) Int. Cl. A61B 5/1455; A61B 5/145; A61B 5/00

(52) U.S. Cl. CPC A61B 5/1455; A61B 5/7203; A61B 5/1451; A61B 2562/0238

(57) ABSTRACT

A living body measuring light source system of a living body measurement apparatus outputs light with a power spectrum as measurement light, in which the light intensity of a characteristic bandwidth including a wavelength (a cause of a local small S/N ratio), which is likely to be absorbed in a known light absorption and scattering wavelength characteristic of a measurement-target living body that is a living body as a measurement target, is set to be higher than that of other bandwidths.

### CROSS-REFERENCE

This application claims the benefit of Japanese Patent Application No. 2014-197033, filed on Sep. 26, 2014. The content of the aforementioned application is incorporated herein by reference in its entirety.

### BACKGROUND

1. Technical Field

The present invention relates to a living body measuring light source system or the like that irradiates a living body with light, and optically measures the constituents of the body fluid in the living body.

2. Related Art

As one of non-invasive measurement technologies, there is provided a well-known technology by which near infrared light is guided via an optical fiber and incident to the skin of a subject, and biological constituents are quantitatively analyzed from the absorbance spectrum of diffuse, reflected and scattered light (for example, refer to JP-A-2007-259967).

The fact that the representative biological tissues of a human body have a light absorption and reflection characteristic (hereinafter, which is referred to as a “light absorption and scattering wavelength characteristic”) is disclosed. Specifically, light with a wavelength around 200 nm to 700 nm is considerably absorbed by hemoglobin which is one of the constituents of the body fluid in the living body, and light with the bandwidth of wavelength greater than or equal to 2000 nm is considerably absorbed by water. For this reason, measurement light with a wavelength bandwidth, which is less absorbed by the constituents (for example, hemoglobin) of the body fluid or water in the living body (which is referred to as an “optical window”) and is good in scattering, is selectively used.

An improvement in the S/N ratio (signal-to-noise ratio) of a detection signal is required to improve measurement accuracy. As a countermeasure to improve measurement accuracy, the value of signal is increased, or the value of noise is decreased. In a measurement apparatus that irradiates a subject with measurement light, when laser beams or short-wavelength LED light with only specific short wavelengths are used as measurement light, in regarding to improving the S/N ratio, incident light may be appropriately intensified to the extent that thermal damage or the like is not caused to occur.

However, when a spectral measurement is made (for example, when an absorbance spectrum or the like is measured) over a wide wavelength bandwidth using measurement with a wavelength bandwidth that is wider than those of laser beams or short-wavelength LEDs, that is, using measurement light referred to as “white light”, it is not necessarily good to simply intensify incident light. The reason for this is that when the light absorption and scattering wavelength characteristic of the biological tissues is carefully examined, there are peaks present at several places, and the light absorption and scattering wavelength characteristic is not homogeneous. Naturally, the S/N ratios of wavelengths which are likely to be absorbed are small compared to those of other wavelengths. When the measurement light is intensified to improve locally small S/N ratios of the wavelengths, the entire light intensity (an integrated value of light intensity in a wavelength direction over the entire relatively wide bandwidth including “optical windows”) of the measurement light is increased before the desired improvement is realized. As a result, the light intensity reaches a well-known guideline (for example, JISC7550: photobiological safety of lamp and lamp system, Japanese Industrial Standards Association, 2011) which is set to prevent the occurrence of thermal damage, which is a problem.

### SUMMARY

An advantage of some aspects of the invention is to improve a locally small S/N ratio induced by the light absorption and scattering wavelength characteristic of a measurement-target living body, and to obtain a good S/N ratio over a wider bandwidth.

A first aspect of the invention is directed to a living body measuring light source system that optically measures the constituents of a body fluid in a living body by irradiating the living body with light, in which the living body measuring light source system irradiates the living body with light in which the light intensity of a first bandwidth of the irradiated light, which corresponds to a characteristic bandwidth in a wavelength characteristic of water absorbing or scattering light, is set to be higher than that of a second bandwidth of the irradiated light.

Examples of the body fluid in the living body include a blood fluid, a lymph fluid, a tissue fluid, and the like.

According to the first aspect of the invention, it is possible to improve a small S/N ratio when the irradiated light is not intensified, and to reduce a deviation from the S/N ratio of the wavelength of the second bandwidth (other bandwidths) of the irradiated light by selectively intensifying light components of the first bandwidth of the irradiated light that corresponds to the characteristic bandwidth in which the irradiated light is likely to be absorbed and scattered in the living body. Since the light intensity of only a partial bandwidth (the first bandwidth) is increased, the light intensity of the entire measurement light with which the living body is irradiated is much reduced compared to when the light source is simply intensified such that the light intensity of the entire wavelengths of the measurement light is increased.

As a result, it is possible to improve a local small S/N ratio induced by the light absorption and scattering wavelength characteristic of the measurement-target living body, and to obtain a good S/N ratio over a wider bandwidth.

A relationship between a wavelength that is selectively intensified and intensity to be increased can be appropriately set according to a characteristic of the body fluid in the living body which is a measurement target.

A second aspect of the invention is directed to the living body measuring light source system according to the first aspect of the invention, in which the living body measuring light source system irradiates the living body with light in which the light intensity of a wavelength of 976 nm exceeds 1 times, and is less than or equal to 100 times the light intensity of a wavelength of 800 nm or 1073 nm. Alternatively, the second aspect of the invention is directed to the living body measuring light source system according to the first aspect of the invention, in which the living body measuring light source system irradiates the living body with light in which the light intensity of the first bandwidth including a wavelength of 976 nm exceeds 1 times, and is less than or equal to 100 times the light intensity of the second bandwidth including a wavelength of 800 nm or 1073 nm.

A third aspect of the invention is directed to the living body measuring light source system according to the first aspect of the invention, in which the living body measuring light source system irradiates the living body with light in which the light intensity of a wavelength of 1196 nm exceeds 1 times, and is less than or equal to 100 times the light intensity of a wavelength of 800 nm or 1073 nm. Alternatively, the third aspect of the invention is directed to the living body measuring light source system according to the first aspect of the invention, in which the living body measuring light source system irradiates the living body with light in which the light intensity of the first bandwidth including a wavelength of 1196 nm exceeds 1 times, and is less than or equal to 100 times the light intensity of the second bandwidth including a wavelength of 800 nm or 1073 nm.

A fourth aspect of the invention is directed to the living body measuring light source system according to the first aspect of the invention, in which the living body measuring light source system irradiates the living body with light in which the light intensity of a wavelength of 1453 nm is greater than or equal to 25 times, and is less than or equal to 100 times the light intensity of a wavelength of 800 nm or 1073 nm. Alternatively, the fourth aspect of the invention is directed to the living body measuring light source system according to the first aspect of the invention, in which the living body measuring light source system irradiates the living body with light in which the light intensity of the first bandwidth including a wavelength of 1453 nm is greater than or equal to 25 times, and is less than or equal to 100 times the light intensity of the second bandwidth including a wavelength of 800 nm or 1073 nm.

Wavelengths of 976 nm, 1196 nm, and 1453 nm are wavelengths which are likely to be locally absorbed in a light absorption and scattering frequency characteristic of water that takes a large percentage of body fluid. Accordingly, the second to fourth aspects of the invention are effective in measurement using light in a wavelength region in which the ratio of light absorption by water is high.

A fifth aspect of the invention is directed to the living body measuring light source system according to the first aspect of the invention, in which the living body measuring light source system irradiates the living body with light in which the light intensity of a wavelength of 758 nm exceeds 1 times, and is less than or equal to 100 times the light intensity of a wavelength of 732 nm. Alternatively, the fifth aspect of the invention is directed to the living body measuring light source system according to the first aspect of the invention, in which the living body measuring light source system irradiates the living body with light in which the light intensity of the first bandwidth including a wavelength of 758 nm exceeds 1 times, and is less than or equal to 100 times the light intensity of the second bandwidth including a wavelength of 732 nm.

A sixth aspect of the invention is directed to the living body measuring light source system according to the first aspect of the invention, in which the living body measuring light source system irradiates the living body with light in which the light intensity of a wavelength of 924 nm exceeds 1 times, and is less than or equal to 100 times the light intensity of a wavelength of 732 nm. Alternatively, the sixth aspect of the invention is directed to the living body measuring light source system according to the first aspect of the invention, in which the living body measuring light source system irradiates the living body with light in which the light intensity of the first bandwidth including a wavelength of 924 nm exceeds 1 times, and is less than or equal to 100 times the light intensity of the second bandwidth including a wavelength of 732 nm.

Wavelengths of 758 nm and 924 nm are wavelengths which are likely to be absorbed by hemoglobin with oxygen saturation corresponding to venous blood. Accordingly, the fifth or sixth aspect of the invention is effective when blood constituents are measurement targets.

These aspects of the invention can be further optimized according to a relationship between the position of incidence of measurement light and the position of reception of the measurement light.

For example, it is preferable to configure, as a seventh aspect of the invention, a measurement apparatus including the living body measuring light source system according to any one of the second, fifth, and sixth aspects of the invention, in which the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 100 mm.

It is preferable to configure, as an eighth aspect of the invention, a measurement apparatus including the living body measuring light source system according to any one of the second, fifth, and sixth aspects of the invention, in which the measurement apparatus measures the constituents of a body fluid in a living body by using reflection type optical measurement in which an incidence-to-emittance distance of light irradiated by the living body measuring light source system is determined to a value that is greater than or equal to 1 mm, and is less than or equal to 10 mm.

It is preferable to configure, as a ninth aspect of the invention, a measurement apparatus including the living body measuring light source system according to the third aspect of the invention, in which the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 50 mm.

It is preferable to configure, as a tenth aspect of the invention, a measurement apparatus including the living body measuring light source system according to the third aspect of the invention, in which the measurement apparatus measures the constituents of a body fluid in a living body by using reflection type optical measurement in which an incidence-to-emittance distance of light irradiated by the living body measuring light source system is determined to a value that is greater than or equal to 1 mm, and is less than or equal to 5 mm.

It is preferable to configure, as an eleventh aspect of the invention, a measurement apparatus including the living body measuring light source system according to the fourth aspect of the invention, in which the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 1.5 mm.

It is preferable to configure, as a twelfth aspect of the invention, a measurement apparatus including the living body measuring light source system according to the fourth aspect of the invention, in which the measurement apparatus measures the constituents of a body fluid in a living body by using reflection type optical measurement in which an incidence-to-emittance distance of light irradiated by the living body measuring light source system is determined to a value that is less than or equal to 1 mm.

### BRIEF DESCRIPTION OF THE DRAWINGS

The invention will be described with reference to the accompanying drawings, wherein like numbers reference like elements.

FIG. 1 is a diagram illustrating an example of the configuration of a living body measurement apparatus.

FIG. 2 is a schematic diagram illustrating operational effects of an optical filter.

FIG. 3 is a graph of the absorbance spectrum of water with an optical path length of 1 mm.

FIG. 4 is a graph of the transmittance of water with an optical path length of 1 mm.

FIG. 5 is a graph illustrating an example of a power spectrum of measurement light.

FIG. 6 is a graph illustrating an example of a power spectrum of the measurement light.

FIGS. 7A and 7B are tables illustrating an example of a relationship between an incidence-to-emittance distance and a power ratio required for the measurement light.

FIG. 8 is a graph illustrating an absorbance spectrum of hemoglobin.

FIG. 9 is a graph illustrating an absorbance spectrum of hemoglobin.

FIG. 10 is a graph illustrating an absorbance spectrum of hemoglobin.

FIGS. 11A and 11B are tables illustrating an example of a relationship between an incidence-to-emittance distance and a power ratio required for the measurement light.

FIG. 12 is a diagram illustrating a modification example of the configuration of the living body measurement apparatus.

FIG. 13 is a diagram illustrating another modification example of the configuration of the living body measurement apparatus.

FIG. 14 is a diagram illustrating a still another modification example of the configuration of the living body measurement apparatus.

### DESCRIPTION OF EXEMPLARY EMBODIMENTS

### First Embodiment

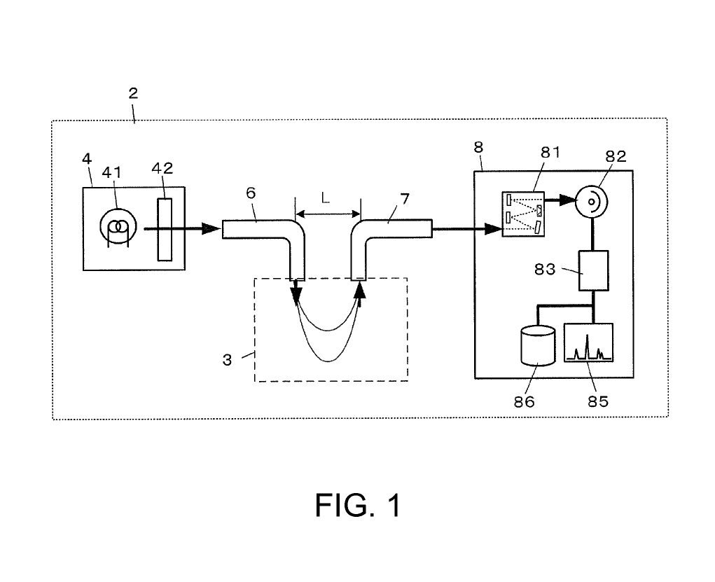

FIG. 1 is a view illustrating an example of the configuration of a living body measurement apparatus in an embodiment.

A living body measurement apparatus 2 in the embodiment is a reflective optical measurement apparatus that irradiates the outer skin of a living body with measurement light, receives a portion of back scattered light (hereinafter, which is referred to as “scattered light”) which returns from the biological tissues to the outer skin, and optically measures the constituents of the body fluids in the living body.

The living body measurement apparatus 2 includes a living body measuring light source system 4 that supplies measurement light; an irradiation optical fiber 6 that guides the measurement light from the living body measuring light source system 4 to a measurement-target living body 3; a light-receiving optical fiber 7 that receives scattered light; and an optical detection device 8 that measures the scattered light guided via the light-receiving optical fiber 7 in a spectroscopic manner.

The irradiation optical fiber 6, the light-receiving optical fiber 7, and the optical detection device 8 can be realized using the well-known measurement apparatuses disclosed in JP-A-2007-259967.

For example, the optical detection device 8 can be realized similarly to a well-known “optical spectrum measurement instrument”. In the embodiment, a monochromator 81 spectrally disperses scattered light guided via the light-receiving optical fiber 7 by wavelength bandwidth of measurement light, and a photometric unit 82 measures the light intensity of each of the spectrally dispersed wavelengths in the form of an electric signal. A signal processing unit 83 appropriately performs digital processes on the electric signals such as amplification or A/D conversion, and calculates a power spectrum of the detected scattered light or a measured value (for example, a glucose level) of the constituents of the body fluid in the living body. The calculated measurement result can be displayed on a display unit 85 such as a flat panel display, and the data of the measurement result can be stored in a storage unit 86 such as an IC memory or a hard disk. Naturally, in addition to these units, the living body measurement apparatus 2 can be appropriately provided with a light guide lens, an optical filter, a shutter mechanism, and the like.

The type of the photometric unit 82 is not limited to a single photoelectric device (for example, a photoelectric tube), and the photometric unit 82 may be a multi-channel charge coupled device (CCD)-type photometric device that can measure a plurality of wavelengths at once. The display unit 85 or the storage unit 86 may be an external device that is connected to and communicated with the living body measurement apparatus 2.

The living body measuring light source system 4 includes a light source unit 41 that emits initial light for the measurement light, and an optical filter 42 that corrects the energy wavelength characteristic (which is a synonym of energy spectral density, and hereinafter, is referred to as a “power spectrum”) of the initial light. Naturally, in addition to these components, a condenser lens and the like can be appropriately mounted in the living body measuring light source system 4.

The light source unit 41 is realized by a single use of or a combination of a halogen lamp, a light-emitting diode (LED), a Xenon lamp, a wide-bandwidth laser beam, or the like. In the embodiment, the initial light is not short-wavelength light, but wide-bandwidth light that is represented by white light. The initial light is a relatively wide bandwidth of light that includes at least a portion of the wavelength bandwidth of the optical window in the known light absorption and scattering wavelength characteristic of the representative biological tissues of the human body. For example, the emission frequency characteristic of a halogen lamp corresponds to the relatively wide bandwidth of frequency characteristic. When an LED is used as a light source, similarly, the relatively wide bandwidth of frequency characteristic may be realized by simultaneously emitting light from a plurality of types of LEDs with a plurality of different emission frequency characteristics.

For example, the optical filter 42 is a dielectric multilayer filter, and the optical filter 42 has 1) an effect of homogenizing and whitening the energy wavelength characteristic of the initial light, and 2) an effect of correcting the light intensity (spectral power) of a local bandwidth, in which there is a local peak of the intensity of absorption and scattering present in the known light absorption and scattering wavelength characteristic of the measurement-target living body 3, to be high relative to those of other bandwidths.

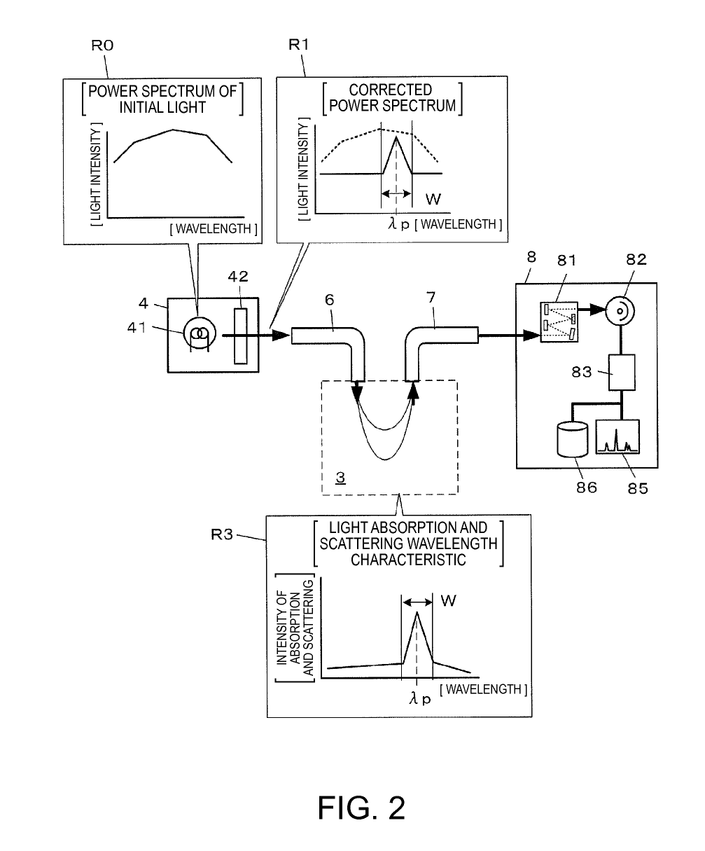

FIG. 2 is a schematic view illustrating the effects of the optical filter 42.

A light absorption and scattering wavelength characteristic R3 which is criteria of the measurement-target living body 3 is already known, and there is a local peak of absorption and scattering present in a wavelength λp. A S/N ratio around the wavelength λp is small. This local bandwidth is referred to as a “characteristic bandwidth W”. A power spectrum R0 of the initial light emitted from the light source unit 41 is already known.

The optical filter 42 homogenizes the power spectrum R0 of the initial light, and corrects the light intensity of the characteristic bandwidth W including the wavelength λp to be higher than that of other bandwidths. Reversely speaking, the light intensity of bandwidths other than the characteristic bandwidth W is decreased. As a result, the measurement light has a power spectrum R1 after being corrected. The local small S/N ratio centered around the concerned wavelength λp is improved by partially increasing the light intensity of the characteristic bandwidth W. Accordingly, the total of light energy of the entire measurement light is limitedly increased, and does not reach a level to cause irradiation-induced thermal damage or the like. As a result, it is possible to improve a local small S/N ratio induced by the light absorption and scattering wavelength characteristic of the measurement-target living body, and to obtain a good S/N ratio over a wider bandwidth.

In FIG. 2, for ease of understanding, the power spectra R0 and R1, and the light absorption and scattering wavelength characteristic R3 are illustrated in a simplified form.

The characteristic bandwidth W, the light intensity of which is increased, preferably is in the range of a wavelength (as illustrated) corresponding to both skirts of a local peak of absorption and scattering in the power spectrum R0, and when the bandwidth is set to at least 40 nm centered around the wavelength λp, it is possible to obtain an advantage of some aspects of the invention.

Hereinafter, as a specific example of the power spectrum R1 of measurement light which is targeted to be obtained in regards to manufacturing the living body measuring light source system 4, a case in which the constituents of the body fluid in the living body are measured using a light intensity of 800 nm to 1800 nm of scattered light is described.

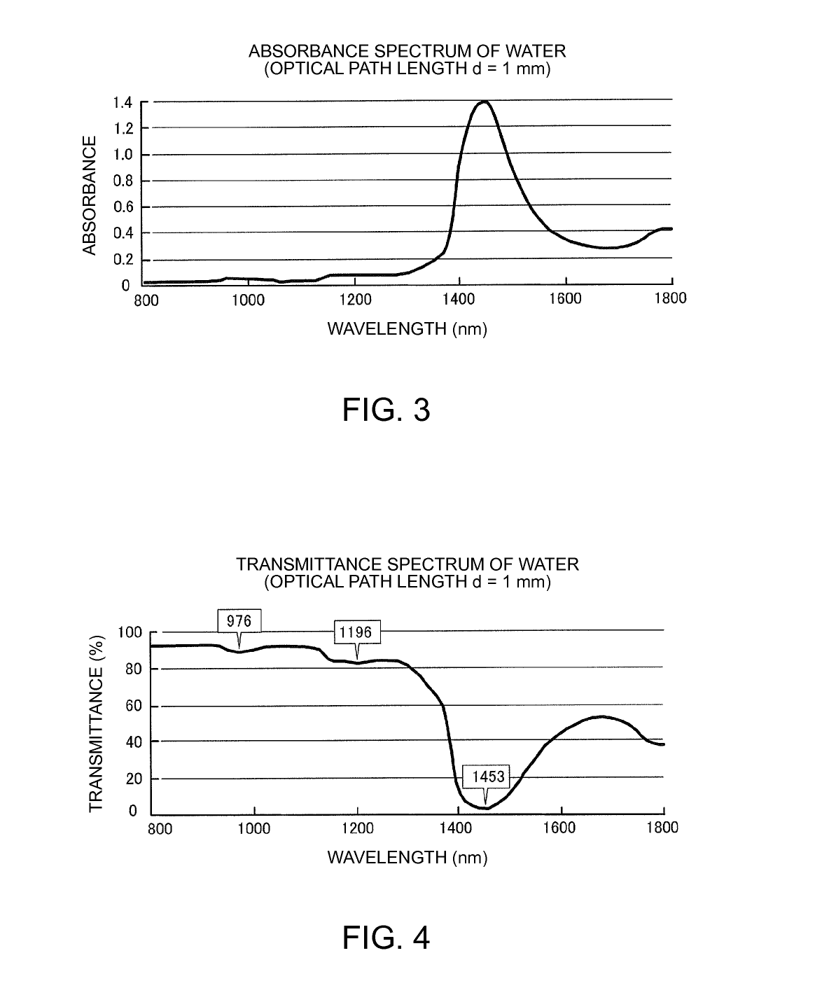

FIG. 3 is a graph illustrating an example of actual measurement of an absorbance spectrum of water with an optical path length of 1 mm. FIG. 4 is a graph illustrating the transmittance of the water. It is understood from these graphs that there are wavelength bandwidths, that is, “characteristic bandwidths” present around wavelengths of 960 nm, 1200 nm, and 1450 nm at which a large amount of the scattered light is absorbed by the water. When the wavelength characteristic of the measurement light is relatively flat, a small amount of scattered light is detected in these characteristic bandwidths compared to scattered light with other wavelengths. That is, the S/N ratios are locally small in the characteristic bandwidths. In order to improve the local small S/N ratio, the power spectrum of the measurement light is set in such a way that the S/N ratio approximately becomes equal in a wavelength bandwidth of 800 nm to 1800 nm.

When the power spectrum of the measurement light is P (λ), and satisfies Expression (1) below, the wavelength dependence of the measurement light approximately becomes flat, and variations in S/N ratio associated with wavelength are decreased. Here, Tw (λ) is the transmittance spectrum of water, Aw (λ) is the absorbance of water, μaw (X) is the absorption coefficient of water, d is an optical path length, and C is a constant.

$$P ( λ ) = \frac{C}{{T}_{w} ( λ )} = \frac{C}{{10}^{- {A}_{w} ( λ )}} = \frac{C}{exp [ - {μ}_{aw} ( λ ) d ]} \qquad ( 1 )$$

Expression (1) can be modified to Expression (2).

ln [P(λ)]=ln C+μ_{aw}(λ)d (2)

It is possible to obtain Expression (3) from Expression (1) based on a relationship in power ratio between wavelengths.

$$\begin{matrix}\frac{P ( {λ}_{2} )}{P ( {λ}_{1} )} = \frac{exp [ - {μ}_{aw} ( {λ}_{1} ) d ]}{exp [ - {μ}_{aw} ( {λ}_{2} ) d ]} \\ = exp \{ [ {μ}_{aw} ( {λ}_{2} ) d - {μ}_{aw} ( {λ}_{1} ) d ] \} \\ = {\{ exp [ {μ}_{aw} ( {λ}_{2} ) - {μ}_{aw} ( {λ}_{1} ) ] \}}^{d}\end{matrix} \} \qquad ( 3 )$$

The living body measuring light source system 4 may be manufactured by determining the power spectrum R0 of the initial light from the light source unit 41, and the specification of the optical filter 42 based on these expressions.

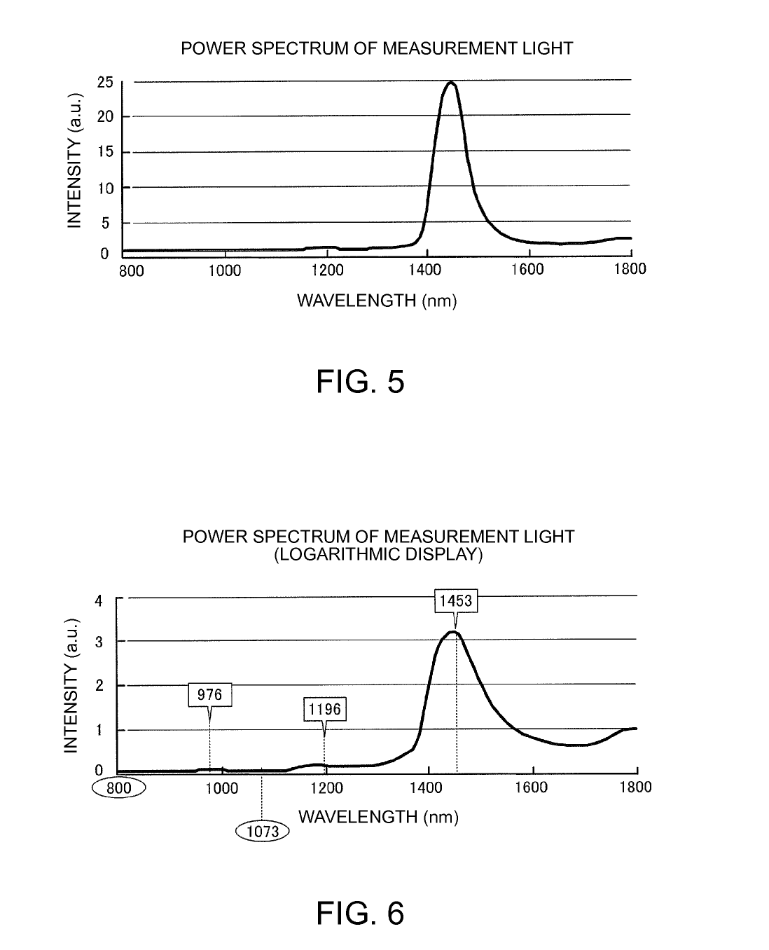

For example, FIG. 5 is a graph illustrating P (λ) when C is equal to 1 and d is equal to 1, and illustrates a power spectrum of measurement light which is targeted to be obtained. FIG. 6 is a graph illustrating 1n (P (λ)) under the same conditions. When measurement light with the power spectrum illustrated by these graphs is realized, the light intensity of each of three characteristic regions including wavelengths of 976 nm, 1196 nm, and 1453 nm is increased compared to that in other bandwidths (bandwidths in which the absorption coefficient and absorbance are low, and the S/N ratio is originally good, and for example, bandwidths around wavelengths of 800 nm and 1073 nm). Accordingly, it is possible to appropriately prevent the occurrence of thermal damage to the living body, and to efficiently realize the measurement of the living body with a low noise level.

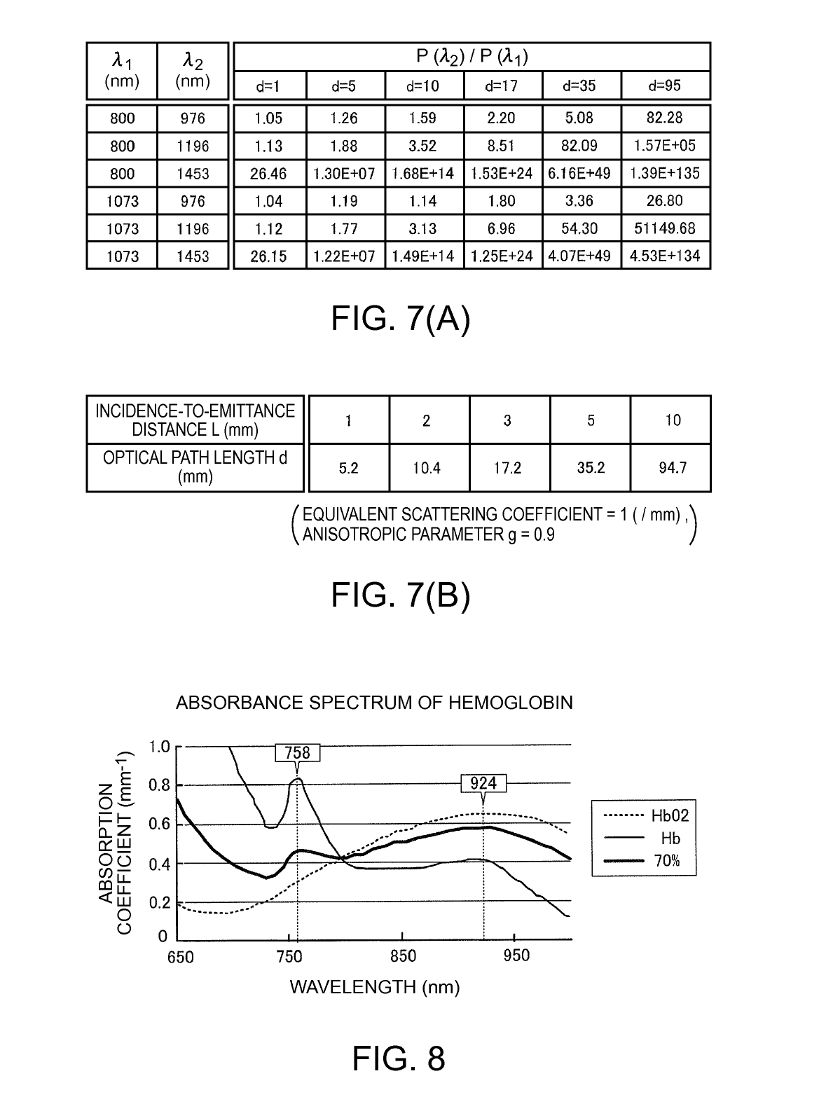

For example, FIGS. 7A and 7B illustrate an example of a relationship between the position of incidence of the measurement light and the position of reception of the scattered light, that is, a relationship between an incidence-to-emittance distance L (refer to FIG. 1) and a power ratio. FIG. 7A is obtained based on the conditions that an approximate relationship between the average optical path length d and the incidence-to-emittance distance L is as illustrated in FIG. 7B. When the power spectrum R0 of the initial light from the light source unit 41 and the specification of the optical filter 42 are determined, if it is not possible to realize the power spectrum with exactly the same characteristic as illustrated in FIGS. 5 and 6, but the relationship between the power ratio and the incidence-to-emittance distance illustrated in FIG. 7A is satisfied by intensifying one of a first wavelength λ₁ and a second wavelength λ₂, and setting the other wavelength to have an originally small S/N ratio, an advantage of some aspects of the invention is obtained.

Specifically, when the incidence-to-emittance distance is determined to be a value which is greater than or equal to 1 mm, and is less than or equal to 10 mm, the light intensity of a wavelength of 976 nm may exceed 1 times, and be less than or equal to 100 times the light intensity of a wavelength of 800 nm or 1073 nm. In transmittance type optical measurement in which measurement light transmits through the measurement-target living body 3 while the measurement-target living body 3 is interposed between the position of incidence of the measurement light and the position of reception of scattered light, when the incidence-to-emittance distance is determined to be a value which is greater than or equal to 1 mm, and is less than or equal to 100 mm, the same power ratio can be applied.

When the incidence-to-emittance distance is determined to be a value which is greater than or equal to 1 mm, and is less than or equal to 5 mm, the light intensity of a wavelength of 1196 nm exceeds 1 times, and be less than or equal to 100 times the light intensity of a wavelength of 800 nm or 1073 nm. In the transmittance type optical measurement in which the incidence-to-emittance distance is determined to be a value which is greater than or equal to 1 mm, and is less than or equal to 50 mm, the same power ratio can be applied.

When the incidence-to-emittance distance is determined to be a value which is less than or equal to 1 mm, the light intensity of a wavelength of 1453 nm is greater than or equal to 25 times, and be less than or equal to 100 times the light intensity of a wavelength of 800 nm or 1073 nm. In the transmittance type optical measurement in which the incidence-to-emittance distance is determined to be a value which is greater than or equal to 1 mm, and is less than or equal to 1.5 mm, the same power ratio can be applied.

Hereinafter, as a specific example of the power spectrum R1 of measurement light which is targeted to be obtained in regards to manufacturing the living body measuring light source system 4, a case in which the constituents of the body fluid in the living body are measured using scattered light with a light intensity less than or equal to 800 nm is described.

In a wavelength bandwidth less than or equal to 800 nm, absorption by hemoglobin is dominant. Accordingly, the absorption coefficient of hemoglobin may be applied to Expressions (2) and (3) in this wavelength bandwidth.

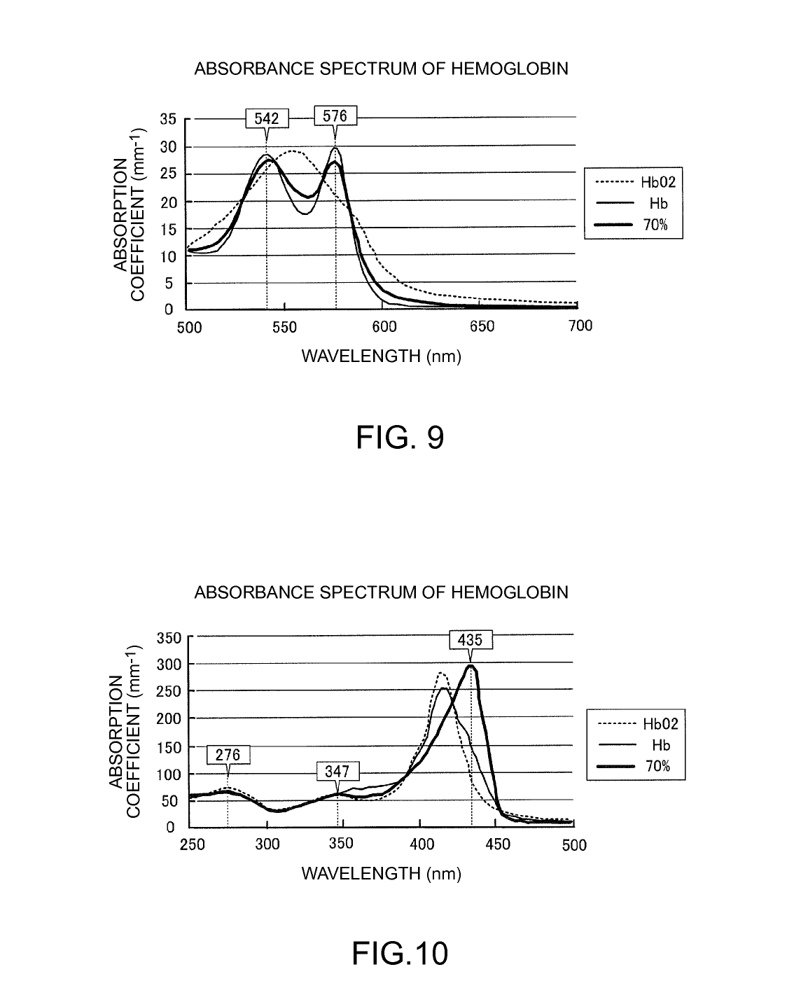

For example, FIGS. 8 to 10 are graphs illustrating absorption spectra of oxygenated hemoglobin (symbol legend: HbO2), deoxygenated hemoglobin (symbol legend: Hb), and a mixture of 70% oxygenated hemoglobin and 30% deoxygenated hemoglobin (symbol legend: 70%). The oxygenated hemoglobin corresponds to arterial blood, and a mixture of 70% oxygenated hemoglobin and 30% deoxygenated hemoglobin corresponds to venous blood. Since the living body measurement apparatus 2 irradiates the skin surface with measurement light, a vein positioned closer to the skin than an artery is assumed to be a target, and attention is paid to the wavelength characteristic of a mixture of 70% oxygenated hemoglobin and 30% deoxygenated hemoglobin.

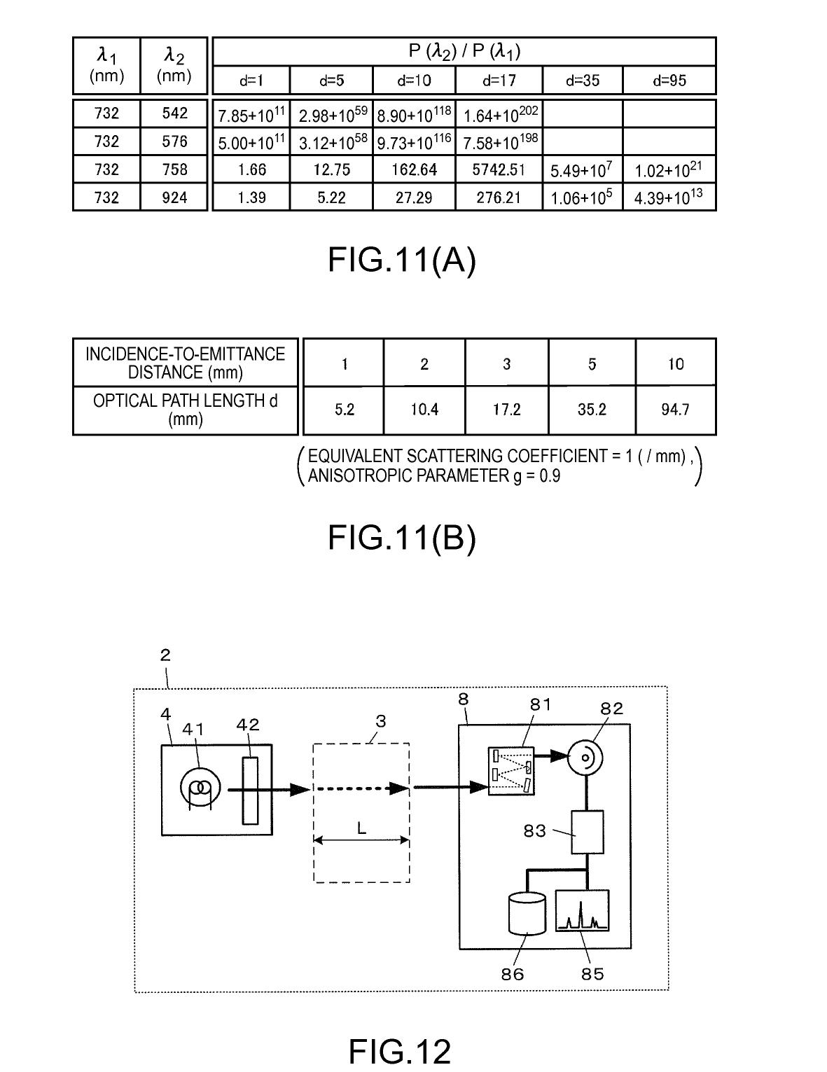

Wavelengths are likely to be absorbed in “characteristic bandwidths” around a wavelength of 924 nm, 758 nm, 576 nm, 542 nm, 435 nm, 347 nm, and 276 nm. As a result, the living body measuring light source system 4 may be manufactured by determining the specifications of the light source unit 41 and the optical filter 42 so as to satisfy the power ratio in FIG. 11A in these characteristic bandwidths.

Specifically, when the incidence-to-emittance distance is determined to be a value which is greater than or equal to 1 mm, and is less than or equal to 10 mm, the light intensity of a wavelength of 758 nm exceeds 1 times, and be less than or equal to 100 times the light intensity of a wavelength of 732 nm. The light intensity of a wavelength of 924 nm may exceed 1 times, and be less than or equal to 100 times the light intensity of a wavelength of 732 nm. In the transmittance type optical measurement in which the incidence-to-emittance distance is determined to be a value which is greater than or equal to 1 mm, and is less than or equal to 100 mm, the same power ratio can be applied.

### MODIFICATION EXAMPLE

An example of the living body measurement apparatus in the embodiment of the invention has been described above; however, the invention is not limited to the aforementioned embodiment, and configuration elements can be appropriately added, omitted, or changed.

### Modification Example 1

For example, the power spectrum of measurement light which is targeted to be obtained is not limited to the power spectrum which is obtained from Expression (1) as illustrated in FIGS. 5 and 6, and when the power spectrum has a characteristic proximate thereto, it is possible to expect the same effect.

For example, when a predetermined bandwidth (for example, approximately 40 nm) centered around a wavelength (for example, 435 nm, 976 nm, or 1196 nm) corresponding to a local small S/N ratio is set, and the bandwidth satisfies a power ratio obtained from Expression (3), it is possible to expect satisfactory effects. In this case, the width of the characteristic bandwidth W can be changed according to the width of a peak of a spectrum that is produced by a wavelength with a small S/N ratio.

### Modification Example 2

It may be considered that independent of Expressions (1) to (3), the vertical axis of the known absorbance spectrum (refer to FIGS. 3, and 8 to 10) of the measurement-target living body 3 is replaced with an axis for the target value of intensification of the wavelength, and the light source unit 41 and the optical filter 42 are set.

### Modification Example 3

When the living body measurement apparatus 2 measures the constituents of the body fluid in the living body by using transmittance type optical measurement in which the incidence-to-emittance distance of measurement light irradiated by the living body measuring light source system 4 is determined to a value that is greater than or equal to 1 mm, and is less than or equal to 1.5 mm, that is, when the living body measuring light source system 4 irradiates the measurement-target living body 3 with measurement light, and the optical detection device 8 receives scattered light from the measurement-target living body 3, as illustrated in FIG. 12, it is possible to omit the irradiation optical fiber 6 and the light-receiving optical fiber 7.

### Modification Example 4

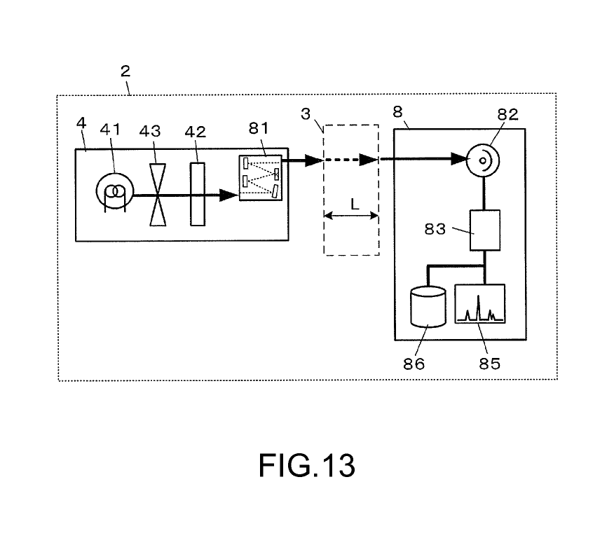

As illustrated in FIG. 13, the monochromator 81 may be provided at the final stage (rear stage of the optical filter 42) of the living body measuring light source system 4 instead of being provided in the optical detection device 8. A light quantity adjustment device 43 may be added between the light source unit 41 and the optical filter 42.

### Modification Example 5

In the embodiment, the light energy of the characteristic bandwidth is set to be higher than that of other wavelengths by the spectrum correction effect of the optical filter 42; however, this setting may be realized by a combination of light beams.

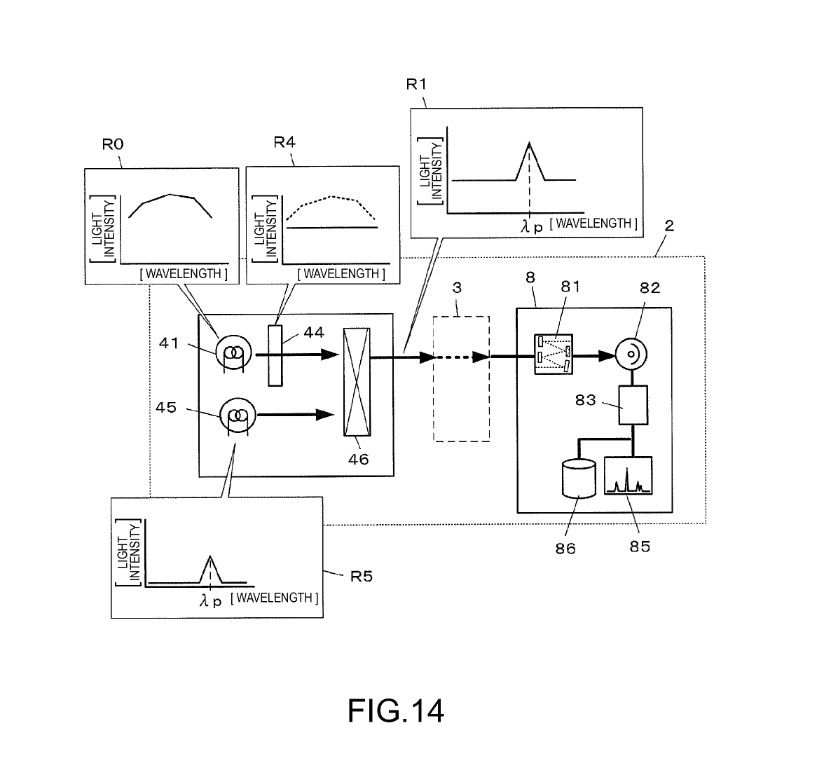

For example, as illustrated in FIG. 14, the power spectrum of initial light emitted from the light source unit 41 is flattened by a whitening filter 44. In addition to the whitening filter 44, a special light source unit 45 (for example, a near infrared LED) that selectively emits light with characteristic bandwidths of wavelengths, and a photomixer 46 are added. The photomixer 46 may mix light passing through the whitening filter 44 with light from the special light source unit 45, and output the resultant light as measurement light.

What is claimed is:

1. A living body measuring light source system that optically measures the constituents of a body fluid in a living body by irradiating the living body with light,
wherein the living body measuring light source system irradiates the living body with light in which the light intensity of a first bandwidth of the irradiated light, which corresponds to a characteristic bandwidth in a wavelength characteristic of water absorbing or scattering light, is set to be higher than that of a second bandwidth of the irradiated light.

2. The living body measuring light source system according to claim 1,
wherein the living body measuring light source system irradiates the living body with light in which the light intensity of the first bandwidth including a wavelength of 976 nm exceeds 1 times, and is less than or equal to 100 times the light intensity of the second bandwidth including a wavelength of 800 nm or 1073 nm.

3. The living body measuring light source system according to claim 1,
wherein the living body measuring light source system irradiates the living body with light in which the light intensity of the first bandwidth including a wavelength of 1196 nm exceeds 1 times, and is less than or equal to 100 times the light intensity of the second bandwidth including a wavelength of 800 nm or 1073 nm.

4. The living body measuring light source system according to claim 1,
wherein the living body measuring light source system irradiates the living body with light in which the light intensity of the first bandwidth including a wavelength of 1453 nm is greater than or equal to 25 times, and is less than or equal to 100 times the light intensity of the second bandwidth including a wavelength of 800 nm or 1073 nm.

5. The living body measuring light source system according to claim 1,
wherein the living body measuring light source system irradiates the living body with light in which the light intensity of the first bandwidth including a wavelength of 758 nm exceeds 1 times, and is less than or equal to 100 times the light intensity of the second bandwidth including a wavelength of 732 nm.

6. The living body measuring light source system according to claim 1,
wherein the living body measuring light source system irradiates the living body with light in which the light intensity of the first bandwidth including a wavelength of 924 nm exceeds 1 times, and is less than or equal to 100 times the light intensity of the second bandwidth including a wavelength of 732 nm.

7. A measurement apparatus comprising:
the living body measuring light source system according to claim 2,
wherein the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 100 mm.

8. A measurement apparatus comprising:
the living body measuring light source system according to claim 5,
wherein the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 100 mm.

9. A measurement apparatus comprising:
the living body measuring light source system according to claim 6,
wherein the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 100 mm.

10. A measurement apparatus comprising:
the living body measuring light source system according to claim 2,
wherein the measurement apparatus measures the constituents of a body fluid in a living body by using reflection type optical measurement in which an incidence-to-emittance distance of light irradiated by the living body measuring light source system is determined to a value that is greater than or equal to 1 mm, and is less than or equal to 10 mm.

11. A measurement apparatus comprising:
the living body measuring light source system according to claim 5,
wherein the measurement apparatus measures the constituents of a body fluid in a living body by using reflection type optical measurement in which an incidence-to-emittance distance of light irradiated by the living body measuring light source system is determined to a value that is greater than or equal to 1 mm, and is less than or equal to 10 mm.

12. A measurement apparatus comprising:
the living body measuring light source system according to claim 6,
wherein the measurement apparatus measures the constituents of a body fluid in a living body by using reflection type optical measurement in which an incidence-to-emittance distance of light irradiated by the living body measuring light source system is determined to a value that is greater than or equal to 1 mm, and is less than or equal to 10 mm.

13. A measurement apparatus comprising:
the living body measuring light source system according to claim 3,
wherein the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 50 mm.

14. A measurement apparatus comprising:
the living body measuring light source system according to claim 3,
wherein the measurement apparatus measures the constituents of a body fluid in a living body by using reflection type optical measurement in which an incidence-to-emittance distance of light irradiated by the living body measuring light source system is determined to a value that is greater than or equal to 1 mm, and is less than or equal to 5 mm.

15. A measurement apparatus comprising:
the living body measuring light source system according to claim 4,
wherein the measurement apparatus measures the constituents of a body fluid in a living body by using transmittance type optical measurement in which an incidence-to-emittance distance of light irradiated by the living body measuring light source system is determined to a value that is greater than or equal to 1 mm, and is less than or equal to 1.5 mm.

16. A measurement apparatus comprising:
the living body measuring light source system according to claim 4,
wherein the measurement apparatus measures the constituents of a body fluid in a living body by using reflection type optical measurement in which an incidence-to-emittance distance of light irradiated by the living body measuring light source system is determined to a value that is less than or equal to 1 mm.
